(19) United States
(12) Patent Application Publication
(10) Pub. No.: US 2005/0287214 A1
(43) Pub. Date: Dec. 29, 2005

(54) Squeeze controlled oral dosage form

(76) Inventors: Atul D. Ayer, Palo Alto, CA (US); Christine Loehrlein, Alameda, CA (US); Scott Gilbert, Menlo Park, CA (US); Liang Chang Dong, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/149,056

(22) Filed: Jun. 8, 2005

Related U.S. Application Data
(60) Provisional application No. 60/583,766, filed on Jun. 28, 2004.

(51) Int. Cl. A61K 9/24

(52) U.S. Cl. 424/473

(57) ABSTRACT

A controlled release dosage form includes an active agent composition and a squeeze layer circumscribing at least a portion of the active agent composition. The squeeze layer includes a material that changes shape in a selected fluid environment of use. The material when it changes shape applies a squeeze force to the active agent composition.

### CROSS-REFERENCE TO RELATED APPLICATION

This application claims benefit, under 35 U.S.C 119(e), of U.S. Ser. No. 60/583,766, filed Jun. 28, 2004, which is incorporated herein by reference.

### BACKGROUND OF INVENTION

The invention relates generally to dosage forms capable of providing controlled release of soluble and poorly soluble active agents.

Controlled release dosage forms that release an active agent from a core site within a tablet are known in the art. The most successful of these dosage forms employ osmosis to provide controlled delivery of the active agent. One classic example of an osmotically-controlled oral dosage form is the OROS® Push-Pull system, available from Alza Corporation, Mountain View, Calif. FIG. 1 shows a basic structure of the Push-Pull system (100). The system 100 includes an osmotic core 102 bounded by a semipermeable membrane 104 having a delivery orifice 106. The osmotic core 102 includes a volume of active agent composition 108 and a volume of expandable composition 110. The osmotic core 102 “pulls” aqueous fluid through the semipermeable membrane 104, and the aqueous liquid hydrates the expandable composition 110. As the expandable composition 110 is hydrated, it expands and “pushes” the active agent in the active agent composition 108 through the delivery orifice 106. In order to achieve a desired release rate or release profile, the active agent composition 108 may incorporate multiple formulation layers containing varying concentrations of active agent.

Prior to hydration, the expandable composition typically accounts for one-third or more of the volume of the osmotic core. To provide a desired dose of an active agent over a time period, the dosage form may have to be so large that patients in need are unwilling or unable to swallow them. This is especially a concern where the dose of the active agent is high and the aqueous solubility of the active agent is low. Moreover, fabrication of known osmotically-controlled dosage forms can be complex and often requires specialized manufacturing machinery, particularly where the active agent composition must be formulated with multiple layers in order to achieve a desired release rate profile. Hence, providing a dosage form with relatively higher active agent loading efficiency would be beneficial since it would allow a desired dose of active agent to be delivered to a subject using a relatively smaller dosage form. In addition, as the size of the dosage form decreases, the ease with which the dosage form can be administered increases and the cost of manufacturing the dosage form decreases.

### SUMMARY OF INVENTION

One aspect of the invention relates to a controlled release dosage form which comprises an active agent composition and a squeeze layer circumscribing at least a portion of the active agent composition, the squeeze layer comprising a material that changes shape in a selected fluid environment of use, wherein the material when it changes shape applies a squeeze force to the active agent composition.

Another aspect of the invention relates to a method of delivering an active agent to a fluid environment of use, comprising placing a controlled release dosage form in the fluid environment of use, the controlled release dosage form comprising an active agent composition including the active agent and a squeeze layer circumscribing at least a portion of the active agent composition, the squeeze layer comprising a material that changes shape in the fluid environment of use, wherein the material when it changes shape generates a squeeze force; hydrating the active agent composition such that a wet mass is formed in which the active agent either dissolves or suspends; and applying the squeeze force to the active agent composition to deliver the active agent.

Other features and advantages of the invention will be apparent from the following description.

### BRIEF DESCRIPTION OF DRAWINGS

FIG. 1 is a cross-section of a prior-art osmotically-controlled oral dosage form.

FIG. 2A is a schematic of a dosage form according to one embodiment of the invention.

FIG. 2B is a cross-section of the dosage form of FIG. 2A.

FIG. 2C is a schematic of a dosage form according to another embodiment of the invention.

FIG. 2D is a cross-section of the dosage form of FIG. 2C.

FIGS. 3A and 3B show cumulative release and release rate, respectively, for a dosage form having the general structure shown in FIGS. 2A and 2B and containing a low solubility active agent.

FIGS. 4A and 4B are graphs showing cumulative release and release rate, respectively, as a function of squeeze band size for a dosage form having the general structure shown in FIGS. 2C and 2D and containing a low solubility active agent.

FIGS. 5A and 5B are graphs showing cumulative release and release rate, respectively, as a function of membrane permeability for a dosage form having the general structure shown in FIGS. 2C and 2D and containing a low solubility active agent.

FIGS. 6A and 6B are graphs showing cumulative release and release rate, respectively, as a function of delivery orifice number and size for a dosage form having the general structure shown in FIGS. 2C and 2D and containing a low solubility active agent.

FIGS. 7A and 7B are graphs showing cumulative release and release rate, respectively, as a function of squeeze band size for a dosage form having the general structure shown in FIGS. 2C and 2D and containing a low solubility active agent.

FIGS. 8A and 8B are graphs showing cumulative release and release rate, respectively, as a function of band size for a dosage form having the general structure shown in FIGS. 2C and 2D and containing a high solubility active agent.

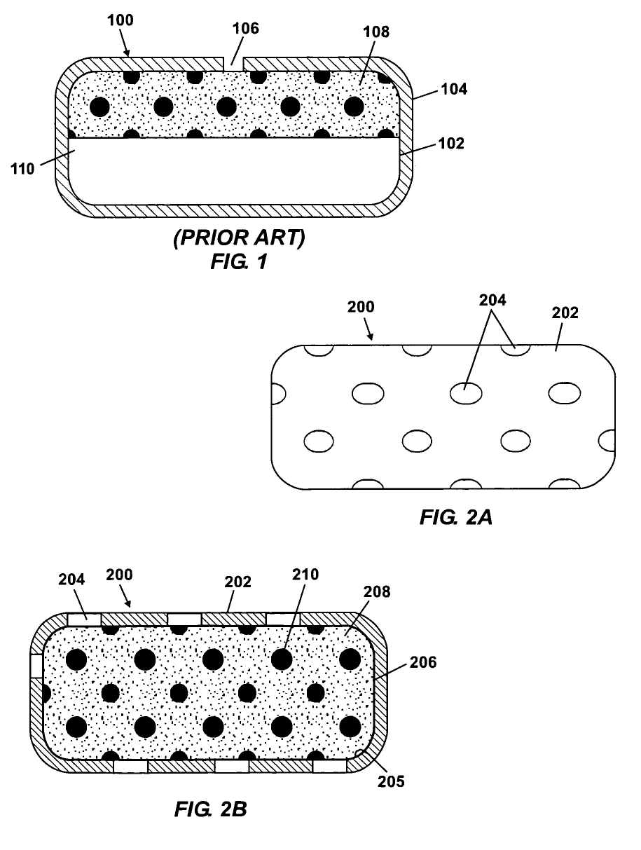

### DETAILED DESCRIPTION OF PREFERRED EMBODIMENTS

The invention will now be described in detail with reference to a few preferred embodiments, as illustrated in accompanying drawings. In the following description, numerous specific details are set forth in order to provide a thorough understanding of the invention. However, it will be apparent to one skilled in the art that the invention may be practiced without some or all of these specific details. In other instances, well-known features and/or process steps have not been described in detail in order to not unnecessarily obscure the invention. The features and advantages of the invention may be better understood with reference to the drawings and discussions that follow. All publications cited herein are incorporated by reference as if reproduced fully herein.

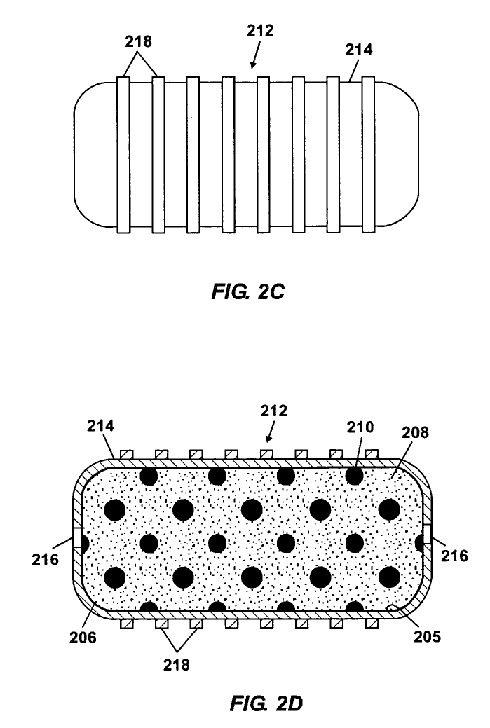

In accordance with one embodiment of the invention, FIG. 2A shows a controlled release dosage form 200 that may be administered orally. The dosage form 200 includes a squeeze layer 202. The squeeze layer 202 is in the form of a bag having openings (or perforations) 204. FIG. 2B shows that the squeeze bag 202 forms a compartment 206 that is occupied by an active agent composition 208. The squeeze bag 202 is made of a material that changes shape when in a selected fluid environment of use. The selected fluid environment of use could be within a human or animal body or could be other fluid environment where controlled delivery of an active agent is desired. When in the selected fluid environment of use, the squeeze bag 202 contracts and exerts a squeeze force on the active agent composition 208. The squeeze bag 202 material is sensitive to a property or condition of the selected fluid environment of use. For example, the material may be sensitive to temperature or pH of the fluid environment or biochemical materials, e.g., enzyme, in the fluid environment. For example, the material could change shape at or near physiological temperature, i.e., 37° C.±1° C., or at physiological pH, e.g., pH 1 to 8, or pH 4 to 8, or pH 5 to 7. In one embodiment, the squeeze bag 202 material is selected from biocompatible, non-erodible, temperature-sensitive materials having elastic properties, such as cross-linked polymers and elastomers, such as thermoplastic elastomers, shape memory polymers, and their copolymers. Examples of these materials include, but are not limited to, silicones (polydimethylsiloxane), polyisoprene (natural rubber), polybutadiene, polyisobutylene, butyl rubber, polychloroprene, epichlorohydrin rubbers, polyurethane thermoplastic elastomer, styrenic thermoplastic elastomer, e.g., Kraton, and ethylene propylene rubber, ethylene propylene diene.

When the dosage form 200 is in a fluid environment of use the squeeze bag 202 contracts and exerts a squeeze force on the active agent composition 208. At the same time, aqueous fluid from the environment of use diffuses into the active agent composition 208 through the openings 204 in the squeeze bag 202, gradually hydrating the active agent composition 208 to form a wet mass in which the active agent 210 either dissolves or suspends. With time, the continued pressure applied to the active agent composition 208 by the squeeze bag 202 squeezes the active agent 210 through the openings 204 into the environment of use. Because the force required to push the active agent 210 out of the compartment 206 is provided by the squeeze bag 202, there is no need for a separate expandable composition in the compartment 206. This means that the entire volume of the compartment 206 can be filled with the active agent composition 208, yielding a dosage form 200 with high active agent loading efficiency. To achieve a desired release rate or profile, the active agent composition 208 may incorporate multiple layers containing varying concentrations of active agent.

The release mechanism of the dosage form 200 is dominated by the squeeze force generated by the squeeze bag 202. The squeeze force depends on the tensile properties of the material used in making the squeeze bag 202 and the physical dimensions of the squeeze bag 202. Once a suitable material is selected for the squeeze bag 202, the squeeze force available for squeezing the active agent composition 208 can be controlled by adjusting the thickness of the wall of the squeeze bag 202. In general, the thicker the wall of the squeeze bag 202, the higher the squeeze force, and the more the release rate can be enhanced. The release rate is also proportional to and controlled by the viscosity of the active agent composition 208 and the size of the openings 204 during operation. The size of the openings 204 can be adjusted to yield controlled release of the active agent. Typically, an active agent composition having a high viscosity requires a squeeze bag with small openings, and an active agent composition having a low viscosity requires a squeeze bag with large openings. The openings 204 are preferably uniformly distributed in the squeeze bag 202 to even out the pressure in the compartment 206.

The active agent composition 208 may be initially formulated as a solid or semisolid. In one embodiment, the active agent composition 208 may include the active agent 210 in an amount ranging from 100 ng to 1100 mg. The active agent composition 208 may include any desired active agent 210. In one embodiment, the active agent 210 is selected from pesticides, herbicides, germicides, biocides, algicides, rodenticides, fungicides, insecticides, antioxidants, plant growth promoters, plant growth inhibitors, preservatives, anti-preservatives, disinfectants, sterilization agents, catalysts, chemical reactants, fermentation agents, foods, food supplements, nutrients, cosmetics, drugs, vitamins, sex sterilants, fertility inhibitors, fertility promoters, microorganism attenuators and other agents that benefit from the environment of use. In another embodiment, the active agent 210 is a physiologically or pharmaceutically active substance that produces a local or systemic effect in warm blooded animals. The active agent 210 could be selected from, for example, immunosuppressive and immunoreactive agents, antiviral and antifungal agents, antineoplastic agents, analgesic and anti-inflammatory agents, antibiotics, anti-epileptics, anesthetics, hypnotics, sedatives, antipsychotic agents, neuroleptic agents, antidepressants, anxiolytics, anticonvulsant agents, antagonists, neuron blocking agents, anticholinergic agents and cholinomimetic agents, antimuscaraicic and mucarinic agents, antiadrenergic and antiarrythmies, antihypertensive agents, hormones, and nutrients. A detailed description of these and other active agents that may be included in the active agent composition 208 can be found in Remington's Pharmaceutical Sciences, 18th editions, 1990, Mack Publishing Co., Philadelphia, Pa.

The active agent 210 may be soluble in water. Specific examples of soluble active agents that may be delivered using the dosage form of the invention include, but are not limited to, acebutolol, acetazolamide, acetophenazine, acetyleamatine, acyclovir, albumin, albuterol, amantadine, ambenoium, arailoride, amitriptyline, amoxicillin, ampetameine, ampicillin, anisotropine, arecoline, atenolol, atracurium, atropine, azatadine, bacitracin, belizsepril, benzphetamine, benztropine, beraprost, betamethasone, betaxolol, bleomycine, brompheniramine, buprenorphine, bupropion, buspirone, calcitonin, captopril, carbinoxamine, carboplatin, cefadroxil, cefazolin, cefixime, cefotaxime, cefotetan, cefotixin, deftriaxone, cefuroxime, chlordiazepoxide, chlorphenirainine, chlorpromazine, ciclopirox, cilastatin, cietidine, clidinium, clindamycin, clomipramine, clonidine, clorazepate, codeine, cromolyn, cyclobenzaprine, deprenyl, desipramine, desmopressin, dexamethasone, dezocine, diclofellac, dicyclomine, diethylpropion, diltiazem, diphenhydramine, dipivefrin, disopyramide, dopainine, dotheipin, doxepin, doxorubicin, doxycycline, encainide, ephedrine, epinephrine, epoetin-alpha, ergonovine, erythromycin, estradiol, conjugated estrogens, esterified estrogens, fenfluramine, fentanyl, fluoxetine, fluphenazine, flurazepam, gepirone, glycopyrrolate, granisetron, guaifenesin, guailadrel, guanethidine, hexobendine, hexoprenaline, histidine, homatropine, hydralzine, hydrocodone, hydrocortisone, hydroxychloroquine, hydroxyzine, hyoscyamine, imipramine, indomethacin, ipratropium bromide, isoproterenol, isosorbide, ketorolac, leuprolide, levobunolol, levorphanol, lidocaine, lisinopril, lithium, mecamylamine, mefenamic acid, menotropins, meperidine, mephentermine, metaproterenol, methamphetarnine, methdilazine, methimazole, methotrimeprazine, methscopolamine, methylphenidate, methylprednisolone, metoprolol, metrifonate, metronidazole, mexiletine, midazolam, minocycline, molidone, morphine, moveltipril, nalbuphine, naloxone, naltrexone, naproxen, neostigmine, netilmicin, nicorandil, nitroftuanatoin, norfenefrine, oslalazine, ondansetron, oxybutynin, oxycodone, oxymorphone, oxytetracycline, pamidronate, pancopride, parathyroid hormone, penicillin G, pentostatin, pentoxifylline, pheneizine, phenmetrazine, phenobarbital, phenoxybenzaniine, phentennine, phenylephrine, pilocarpine, pravastatin, probarbital, prochlorperazine, procyclidine, promethazine, propantheline, propiomazine, propranolol, protryptyline, psuedoephedrine, pyridostigmine, quinapril, quinidine, ramoplanin, ranitidine, rilmenidine, ritodrine, saralasin, scopolarnine, sulfadiazine, tacrine, teicoplanin, terazosin, terbutaline, tertatolol, tetracaine, tetracycline, theophylline, thiethylperazine, thioridazine, thiothixene, ticlopidine, timolol, tobramycin, tolmetin, tranyleypromine, trapidil, trifluoperazine, trimeprazine, trimetazidine, trimethobenzamide, triprolidine, tubocurarine, valproic acid, vancomycin, verapamil, warfarin, zidovudine, and soluble derivatives, pro-drugs, isomers, and salts of the above.

The active agent 210 may be poorly soluble in water. Poorly water soluble drugs are those with dose/solubility volume larger than 250 mL. Specific examples of poorly soluble active agents that may be delivered using the dosage form of the invention include, but are not limited to, acetaminophen, acyclovir, amiloride, amlodipine, amoxapine, amoxicillin, ampicillin, aspirin, atenolol, atropine, auranofin, azathioprine, baclofen, benazepril, bendroflumethiazide, bromocriptine, bumetanide, buprenorphine, busulfan, calcitriol, carbamazepine, carbidopa, cefaclor, cephalexin, chlordiazepoxide, chlorpheniramine, chlorpromazine, chlorpropamide, chlorthalidone, chlorzoxazone, cholestyramine, cimetidine, ciprofloxacin, clarithromycin, clemastine, clonazepam, clotrimazole, clozapine, codeine, cyclosporine, chlorothiazide, cyclophosphamide, deserpidine, desogestrel, dexamethasone, dextromethorphan, dicyclomine, diflunisal, digoxin, diphenoxylate, dipyridamole, disopyramide, doxazosin, doxycycline, enalapril, ephedrine, erythromycin, estazolam, estradiol,ethinyl estradiol,etodolac, etoposide, famotidine, felodipine, fenoprofen, fentanyl, finasteride, fluconazole, fludrocortisone, fluoxetine, fluoxymesterone, fluphenazine, flurbiprofen, flutamide, furosemide, ganciclovir, gemfibrizil, glipizide, glyburide, granisetron, guanabenz, haloperidol, homatropine, hydralazine, hydrochlorothiazide, hydrocodone, hydromorphone, hydroxyzine, ibuprofen, isosorbide dinitrate, pseudoephedrine, progesterone, naloxone, imipramine, indapamide, indomethacin, isosorbide, isotretinoin, isradipine, itraconazole, ketoconazole, ketoprofen, levonorgestrel, levorphanol, lidocaine, liothyronine, lisinopril, lithium, lomefloxacin, loperamide, lorazepam, lovastatin, loxapine, maprotiline, meclizine, medroxyprogesteron, mefenamic acid, meperidine, mestranol, methotrexate, methyclothiazide, methylphenidate, methylprediiisolone, methyltestosterone, metolazone, metronidazole, miconazole, minocycline, minoxidil, morphine, nabumetone, nadolol, naltrexone, nicardipine, nicotine, nifedipine, nimodipine, nitrofarantoin, nitroglycerin, norfloxacin, nystatin, octreotide, ofloxacin, orneprazole, oxaprozin, oxazepana, oxycodone, oxyphencyclimine, oxytetracycline, paclitaxel, pararnethasone, paroxetine, pemoline, penicillin, pentaerythritol, pentamidine, pentazocine, pergolide, perphenazine, phenazopyridine, pheneizine, phenobarbitol, phenoxybenzamine, phenytoin, physostigmine, pimozide, pindolol, polythizide, prazepain, prazosin, prednisolone, prednisone, probucol, prochloperazine, procyclidine, propofol, propranolol, propylthiouracil, pyrimethamine, quinidine, raraipril, rescinnamine, reserpine, rifabutin, rifapentine, respiridone, sahneterol, sertraline, siagoside, siravastatin, spironolactone, sucralfate, sulfadiazine, sulfamethoxazole, sulfamethizole, sulindac, sulpiride, tamoxifen, tandospirone, temazepam, terazosin, terbinafine, terconazole, terfenadine, tetracaine, tetracycline, theophylline, thiethylperazine, thioridazine, thiothixene, thyroxine, timolol, topiramate, tranylcypromine, trazodone, tretinoin, triamcinolone, trimethoprim, triazolam, trichlormethiazide, trihexphenidyl, trioxsalen, vinblastine, vitamin B, and insoluble derivatives, pro-drugs, isomers, and salts of the above.

The active agent composition 208 may also include one or more pharmaceutical adjuncts that would facilitate manufacture of the active agent composition 208 and release of the active agent 210 in the environment of use. The various constituents of the active agent composition 208 are formulated such that as the active agent composition 208 absorbs an aqueous fluid in an environment of use, the active agent composition 208 is converted into a wet mass that can be expelled through the openings 204 in the squeeze bag 202. The active agent composition 208 may be formed by compression or molding. A compressed active agent composition 208 may be made by standard techniques such as wet granulation, dry granulation, or direct compression. In wet granulation, for example, the active ingredient and pharmaceutical excipients in the active agent composition 208 are weighed and blended. A liquid binder is then added to the blend to form a damp mass. The damp mass is passed through a screen to prepare granules. The granules are then dried and passed through another screen of smaller mesh size than that used to prepare the granules. Then, a dry lubricant is blended with the granules, and the blend is compressed into a tablet. The shape of the tablet is determined by the die and punches used in the compression. The tablet may have a round, oblong, or other unique shape. To form the dosage form 200, the squeeze bag 202 may be provided in the form of a tube that can be slipped over the active agent composition 208. The ends of the tube may then be sealed around the active agent composition 208.

In order to provide an active agent composition 208 that upon absorption of an aqueous fluid converts to a wet mass in which the active agent 210 either dissolves or suspends, the active agent composition 208 optionally includes a pharmaceutically acceptable hydrophilic polymer. Numerous different hydrophilic polymers are suitable for use in the active agent composition 208. Exemplary hydrophilic polymers include the following, a maltodextrin polymer having the formula (C₆H₁₀O₅)_{λ}·H₂O, wherein λ is 3 to 7,500, and the maltodextrin polymer possesses a 500 to 1,250,000 grams per mole number-average molecular weight; a poly(alkylene oxide), e.g., poly(ethylene oxide) and poly(propylene oxide) having 7,000 to 750,000 number-average molecular weight or, more specifically, a poly(ethylene oxide) having at least one of a 100,000, 200,000, 300,000, or 400,000 number-average molecular weight; an alkali carboxyalkylcellulose having a 10,000 to 175,000 number-average molecular weight, wherein the alkali is sodium, potassium, calcium, or lithium and the alkyl is 1 to 5 carbons, such as a methyl, ethyl, propyl, butyl, or pentyl group; or a copolymer of ethylene-acrylic acid, e.g., methacrylic or ethacrylic acid having a 10,000 to 1,500,000 number-average molecular weight. Non-polymeric compounds, such as monosacharrides and disacharrides, are also suitable for use as hydrogel compounds in the active agent composition 208. The precise amount of hydrophilic polymer included in the active agent composition 208 will vary depending upon, among other factors, the desired viscosity of the active agent composition 208 during operation and the type of active agent 210 to be delivered. Where the active agent composition 208 includes a hydrophilic polymer, the amount of hydrophilic polymer included will preferably range between about 5 mg and 400 mg. Moreover, though the active agent composition 208 may be formulated using a single type of hydrophilic polymer, more than one different type of hydrophilic polymer, including blends of different molecular weight hydrophilic polymers of the same type, may also be used in the active agent composition 208.

The active agent composition 208 may also include a binder. The binder imparts cohesive qualities to the active agent composition 208 and may be provided in a solution form or a dry form to prepare the active agent composition 208. Binders that may be included in the active agent composition 208 include, for example, starch, gelatin, molasses, methylcellulose, hydroxypropylcellulose, hydroxypropyl methylcellulose, and a vinyl polymer exhibiting a 5,000 to 350,000 number-average molecular weight, such as a poly-n-vinylamide, poly-n-vinylacetamide, polyvinyl pyrrolidone (PVP), poly-n-vinylcaprolactone, poly-n-vinyl-5-methyl-2-pyrrolidone, or poly-n-vincylpyrrolidone copolymers with a member selected from, for example, vinyl acetate, vinyl alcohol, vinyl chloride, vinyl fluoride, vinyl butyrate, vinyl laureate, and vinyl stearate. If desired, the active agent composition 208 may include more than one different type of binder. When one or more binders are included in the active agent composition 208, the binder or mixture of binders may represent up to about 100 mg of the active agent composition 208, and preferably between 0.01 mg to 50 mg.

A tableting lubricant may also be included in the active agent composition 208. The tableting lubricant would lessen adhesion of the active agent composition 208 to tooling, such as die walls or punch faces, or machinery used during manufacture of the active agent composition 208. Tableting lubricants suitable for use in the active agent composition 208 include, for example, polyethylene glycol, sodium stearate, oleic acid, potassium oleate, caprylic acid, sodium stearyl famarate, magnesium palmitate, calcium stearate, zinc stearate, magnesium stearate, magnesium oleate, calcium pahnitate, sodium sebacate, potassium laureate, stearic acid, salts of fatty acids, salts of alicyclic acids, salts of aromatic acids, oleic acid, palmitic acid, a mixture of a salt of a fatty, alicyclic, or aromatic acid, and a mixture of magnesium stearate and stearic acid. If included in the active agent composition 208, the tableting lubricant preferably accounts for between about 0.01 mg and 20 mg of the active agent composition 208.

The active agent composition 208 may also include a nontoxic colorant or dye. The colorant may provide the dosage form with a more aesthetically pleasing appearance. Moreover, the colorant may serve to identify the dosage form during manufacture or in anticipation of administration. Colorants suitable for use in the active agent composition include, for example, ferric oxide red, ferric oxide yellow, ferric oxide green, ferric oxide black, FD&C (Food, Drug, and Cosmetic Act) dyes such as Blue #1 (brilliant blue FCF), Green #6 (quinizarine green SS), Red #22 (eosine) and Yellow #8 (uranine), pharmaceutical dyes diluted with aluminum oxide, and the like. The amount of colorant formulated within the active agent composition will depend upon the desired color intensity. Typical levels of use are 0.5 wt % to 2 wt% colorant based on the weight of the material layer into which the colorant is incorporated.

To inhibit oxidation, the active agent composition 208 may also include an antioxidant. The antioxidant slows down or prevents the oxidation of the dosage form and its ingredients by atmospheric oxygen. Representative oxidants that may be included in the active agent composition 208 include, for example, ascorbic acid, fumaric acid, asorbyl palmitate, butylated hydroxyanisole, a mixture of 2 and 3 tertiary-butyl-4-hydroxyanisole, butylated hydroxytoluene, sodium isoascorbate, dihydroguaretic acid, sodium ascorbate, sodium metabisulfite, potassium ascobate, vitamin E, propyl gallate. If used, the antioxidant preferably accounts for up to about 10 mg of the active agent composition 208.

In accordance with another embodiment of the invention, FIG. 2C shows a dosage form 212, which is the dosage form (200 in FIGS. 2A and 2B) modified to include a semipermeable membrane 214. FIG. 2D shows that the semipermeable membrane 214 forms the compartment 206 that is occupied by the active agent composition 208. The semipermeable membrane 214 is permeable to the passage of an aqueous fluid and substantially impermeable to the active agent 210 in the active agent composition 208. The semipermeable membrane 214 includes one or more delivery orifices 216 for delivery of the active agent 210 to an environment of use. Returning to FIG. 2C, a squeeze layer 218 circumscribes portions of the semipermeable membrane 214. The squeeze layer 218 is in the form of bands. In alternate embodiments, the squeeze layer 218 may be provided in the form of a bag with openings or sleeve with or without openings. Where included, the openings should be large enough to avoid interference with operation of the semipermeable membrane 214 and delivery of the active agent through the delivery orifice(s) 216. The squeeze bands 218 are made of a material that changes shape in a fluid environment of use as previously described for the squeeze bag (202 in FIGS. 2A and 2B). One or more squeeze bands 218, preferably 2 to 10 bands, more preferably 4 to 8 bands, may circumscribe the semipermeable membrane 214. The squeeze bands 218 may be spaced apart as shown or placed close together.

When the dosage form 212 is in a fluid environment of use, the squeeze bands 218 contract and exert a squeeze force on the semipermeable membrane 214, which is transferred to the active agent composition 208. At the same time, aqueous fluid permeates the compartment 206 through the semipermeable membrane 214 and hydrates the active agent composition 208 to form a wet mass in which the active agent 210 either dissolves or suspends. The rate at which aqueous fluid permeates the compartment 206 is controlled by the semipermeable membrane 214. With time, the continued pressure applied to the semipermeable membrane 214 and active agent composition 208 by the squeeze bands 218 squeezes the active agent 210 through the delivery orifice(s) 216 into the environment of use. The hydration mechanism of the dosage form 212 is dominated by osmosis. The release rate is controlled by the permeability of the semipermeable membrane 214, the squeeze force generated by the squeeze bands 218, and viscosity of the active agent composition 208 during release.

In one embodiment, the semipermeable membrane 214 is made of a material that is non-toxic to the intended subject and does not affect the performance of the active agent 210 in the active agent composition 208. Preferably, the semipermeable membrane 214 maintains its chemical integrity in the intended environment of use at least for the operational life of the dosage form 212. That is, the semipermeable membrane 214 should not undergo a chemical change as the active agent 210 is dispensed from the dosage form 212, even as the active agent composition 206 disintegrates during delivery of the active agent 208. The semipermeable membrane is preferably collapsible without breaking under the squeeze force applied by the squeeze bands 218. The materials useful for manufacturing the semipermeable membrane 214 are, in a preferred embodiment, non-erodible and are insoluble in fluids.

Typical polymeric materials for forming the semipermeable membrane 214 include cellulose esters, cellulose ethers, or cellulose ester-ethers. Suitable cellulosic polymers have a degree of substitution (D.S.) on their anhydroglucose unit from greater than 0 up to 3, inclusive. The term “degree of substitution,” as used herein, refers to the average number of hydroxyl groups originally present on the anhydroglucose unit comprising the cellulose polymer that are replaced by a substituting group. Representative polymeric materials include a member selected from the group consisting of cellulose acylate, cellulose diacylate, cellulose triacylate, cellulose acetate, cellulose diacetate, cellulose triacetate, mono-, di- and tri-cellulose alkanylates, mono-, di- and tri-cellulose aroylates, and the like. Exemplary polymers include cellulose acetate having a D.S. up to 1 and an acetyl content up to 21%; cellulose acetate having an acetyl content of 32 to 39.8%; cellulose diacetate having a D.S. of 1 to 2 and an acetyl content of 21% to 35%; cellulose triacetate having a D.S. of 2 to 3 and an acetyl content of 35% to 44.85; and the like. Examples of more specific cellulose polymers include cellulose propionate having a D.S. of 1.8 and a propionyl content of 39.2% to 45% and a hydroxyl content of 2.8% to 5.4%; cellulose acetate-butyrate having a D.S. of 1.8 and an acetyl content of 13% to 15% and a butyryl content of 34% to 39%; cellulose acetate butyrate having an acetyl content of 2% to 29%, a butyryl content of 17% to 53% and a hydroxyl content of 0.5% to 4.7%; cellulose triacylates having a D.S. of 2.9 to 3 such as cellulose trivalerate, cellulose trilaurate, cellulose tripalmitate, cellulose trisuccinate, and cellulose trioctanoate; cellulose diacylates having a D.S. of 2.2 to 2.6 such as cellulose disuccinate, cellulose dipalmitate, cellulose dioctanoate, cellulose dipentate, and the like.

Other examples of semipermeable polymers that may be used for manufacturing the semipermeable membrane 214 include, but are not limited to, acetaldehyde dimethyl acetate, cellulose acetate ethylcarbamate, cellulose acetate phthalate for use in environments having a low pH, cellulose acetate methyl carbamate, cellulose acetate dimethylaminoacetate, semipermeable polyamides, semipermeable polyurethanes, semipermeable sulfonated polystyrenes, cross-linked selective semipermeable polymers formed by the coprecipitation of a polyanion and a polycation as disclosed in U.S. Pat. Nos. 3,173,876; 3,276,586; 3,541,005; 3,541,006; and 3,546,142; semipermeable polymers as disclosed by Loeb and Sourirajan in U.S. Pat. No. 3,133,132; lightly cross-linked plasticized polystyrene derivatives; cross-linked poly(sodium styrene sulfonate); cross-linked poly(vinylbenzyltrimethyl ammonium chloride); semipermeable polymers exhibiting a fluid permeability of 10⁻⁵ to 10⁻¹ (cc. mil/cm²·hr·atm) expressed as per atmosphere of hydrostatic or osmotic pressure difference across the semipermeable membrane. The polymers are known to the art in U.S. Pat. Nos. 3,845,770; 3,916,889; and 4,160,020; and in Handbook of Common Polymers by Scott, J. R. and Roff, W. J., (1971) published by CRC Press, Cleveland, Ohio.

In order to achieve a desired osmotic pressure gradient across the semipermeable membrane 214, the active agent composition 208 may include an osmoagent (also known as an osmotically effective compound or an osmotically effective solute). The osmoagent creates an osmotic pressure gradient across the semipermeable membrane 214, causing aqueous fluid from the environment to be taken into the active agent composition 208 occupying the compartment 206. Any compound capable of generating an osmotic pressure gradient across the semipermeable membrane 214 while not adversely affecting the performance or function of the semipermeable membrane 214 and the active agent 210 may be used as an osmoagent in the active agent composition. Examples of osmoagents that may be formulated into the active agent composition 208 include adipic acid, alanine, ammonium phosphate dibasic, arginine, ascorbic acid, boric acid, calcium gluconate, calcium nitrate, citric acid, dextrose, diammonium succinate, disodium adipate, dipotassium adipate, dipotassium succinate, disodium succinate, fructose, fumaric acid, galactose, gluconodeltalactone, glutaric acid, glycine, lactose, lysine, magnesium benzoate, magnesium sulfate, malic acid, maleic acid, mannitol, monosodium glutamate, monopotassium adipate, monosodium adipate, monopotassium succinate, monosodium succinate, potassium bicarbonate, potassium chloride, potassium citrate, potassium phosphate dibasic, potassium phosphate monobasic, dipotassium succinate, potassium sodium bitartrate, potassium sulfate, sodium ascorbate, sodium bicarbonate, sodium carbonate, sodium chloride, sodium citrate, sodium ftimarate, sodium nitrite, sodium glycerophosphate, sodium glycinate, sodium potassium tartrate, sodium EDTA, sodium phosphate dibasic, sodium phosphate monobasic, disodium succinate, sodium phosphate, sodium tartrate sodium bisulfate, sodium bitartrate, sorbitol, succinic acid, sucrose, sucrose acetate isobutyrate, tartaric acid, urea, xylose, xylitol, and blends of two or more selected from the group of these osmoagents. Where included in the active agent composition 208, the precise amount of osmoagent will vary depending on, among other factors, the materials used in both the active agent composition, the amount and type of active agent to be delivered, and the desired release rate of the active agent.

The following examples are illustrative of the invention and are not to be construed as limiting the scope of the invention. In the examples below, active agent release studies were performed using a USP Apparatus 2-type dissolution bath. The dosage form was released into 900 mL of artificial intestinal fluid at pH 6.8 (enzyme-free) and 37° C. with a paddle stir rate of 100 rpm.

### EXAMPLE 1

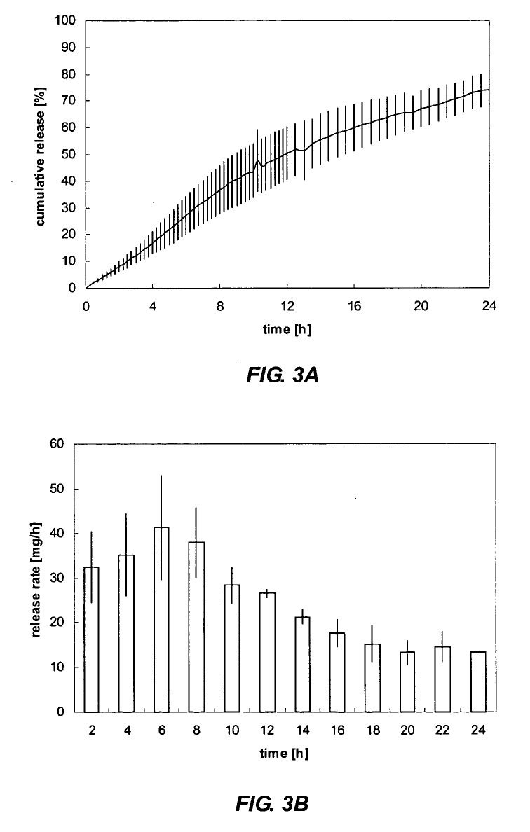

A dosage form having a structure similar to the one shown in FIGS. 2A and 2B was prepared with the formulation shown in Table 1 as the active agent composition. The active agent composition was initially provided in solid form. The squeeze bag enclosing the active agent composition was made of silicone elastomer. The squeeze bag had twenty-eight 40-mm holes, an inner diameter of 1/16 in. (1.59 mm), and an outer diameter of 3/32 in. (2.38 mm). FIGS. 3A and 3B show the cumulative release and release rate, respectively, of the dosage form. FIGS. 3A and 3B show an initial increase in release rate followed by a decreasing tail. The initial increase may result from the squeeze force applied by the squeeze bag. The release rate eventually decreases as the holes shrink with the bag and the squeeze force decreases.

TABLE 1

| | Substance | Wt % |
|---|---|---|
| Active Agent | Acetaminophen (solubility = 20 mg/ml) | 80 |
| Surfactant | Lutrol F127 | 6 |
| Hydrophilic Polymer | Polyox N80 | 10 |
| Binder | PVP K90 | 3 |
| Lubricant | Magnesium Stearate | 1 |

### EXAMPLE 2

Five dosage forms, each having a structure similar to the one shown in FIGS. 2C and 2D, were prepared. Each dosage form had the formulation shown in Table 2 as the active agent composition.

TABLE 2

| | Substance | Wt % |
|---|---|---|
| Active Agent | Acetaminophen (solubility = 20 mg/ml) | 70 |
| Surfactant | Lutrol F127 | 16 |
| Hydrophilic Polymer | Polyox N80 | 10 |
| Binder | PVP K90 | 3 |
| Lubricant | Magnesium Stearate | 1 |

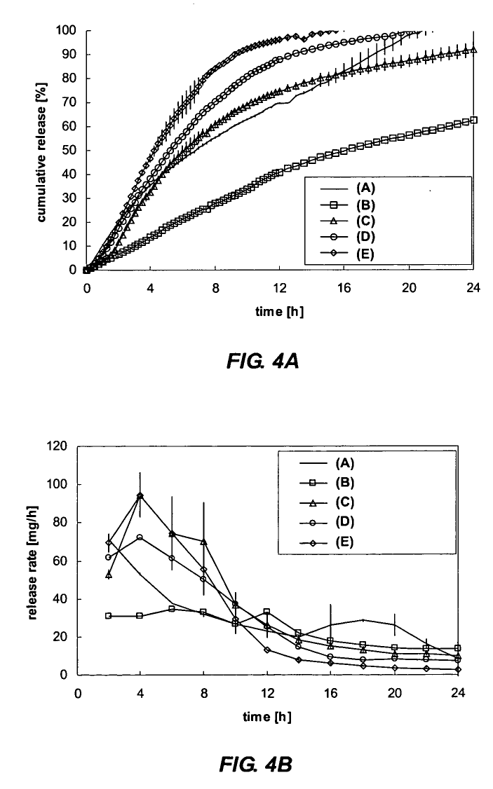

In Example 2, each dosage form had a semipermeable membrane enclosing the active agent composition. The semipermeable membrane in each dosage form had two 40-mm delivery orifices. The semipermeable membrane in each dosage form was composed of 60% by weight Eastman cellulose acetate (CA 398-10) and 40% by weight Lutrol® F68 and had a thickness of 2.5 mm. Four of the dosage forms each had eight squeeze bands circumscribing portions of the semipermeable membrane, and the squeeze bands were made of silicone elastomer. The study evaluated the effect of squeeze band size on release rate. Physical dimensions of the squeeze bands used in the dosage forms are summarized in Table 3 below. FIGS. 4A and 4B show cumulative release and release rate, respectively, of the dosage forms. The results show that thicker walled squeeze bands enhance release rate. The results also show that the inner diameter of the squeeze bands control the residual volume of the dosage form. A smaller inner diameter produces a larger cumulative release or small residual volume.

TABLE 3

| Dosage | Squeeze Band | Squeeze Band | Wall |
|---|---|---|---|
| Form | (Inner Diameter) | (Outer Diameter) | Thickness |
| A | N/A | N/A | N/A |
| B | 1/4 in. (6.35 mm) | 5/16 in. (7.94 mm) | 1.59 mm |
| C | 1/8 in. (3.18 mm) | 1/4 in. (6.35 mm) | 3.17 mm |
| D | 1/16 in. (1.59 mm) | 3/32 in. (2.38) | 0.79 mm |
| E | 1/16 in. (1.59 mm) | 1/8 in. (3.18 mm) | 1.59 mm |

### EXAMPLE 3

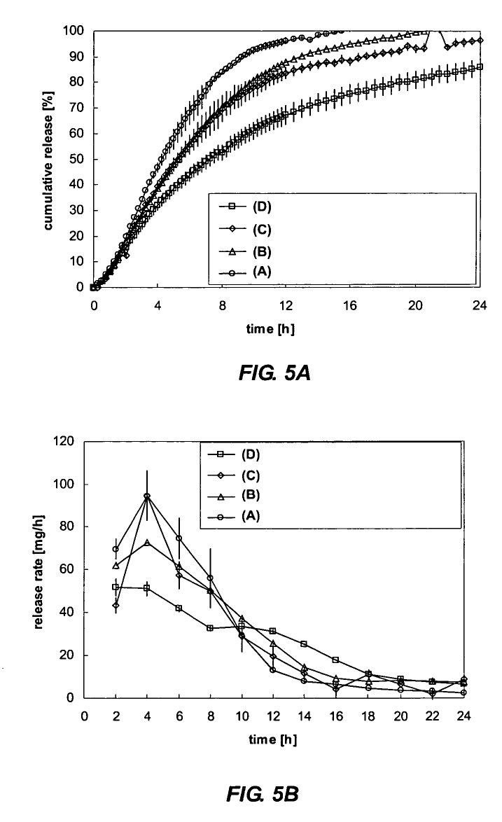

Four dosage forms, each having a structure similar to the one shown in FIGS. 2C and 2D, were prepared. Each dosage form had the formulation shown in Table 2 as the active agent composition. Each dosage form had a semipermeable membrane enclosing the active agent composition. The semipermeable membrane in each dosage form had two 40-mm delivery orifices and a thickness of 2.5 mm. Each dosage form had eight squeeze bands circumscribing portions of the semipermeable membrane, and the squeeze bands were made of silicone elastomer. Additional properties of the semipermeable membrane and squeeze bands used in the dosage forms are summarized in Table 4 below. The study evaluated the effect of membrane permeability on release rate. The cumulative release and release rate, respectively, for the four dosage forms are shown in FIGS. 5A and 5B. The semipermeable composition used in Dosage Forms A and B have a permeability greater than that used in Dosage Forms C and D. The results show that higher flux membranes produce faster release.

TABLE 4

| | | | Squeeze Band |
|---|---|---|---|
| Dosage | Semipermeable | Squeeze Band | (Outer |
| Form | Composition | (Inner Diameter) | Diameter) |
| A | 60% Cellulose Acetate | 1/16 in. | 1/8 in. (3.18 mm) |
| | 40% Lutrol ® F68 | (1.59 mm) | |
| B | 60% Cellulose Acetate | 1/16 in. | 3/32 in. (2.38 mm) |
| | 40% Lutrol ® F68 | (1.59 mm) | |
| C | 70% Cellulose Acetate | 1/16 in. | 1/8 in. (3.18 mm) |
| | 30% Lutrol ® F68 | (1.59 mm) | |
| D | 70% Cellulose Acetate | 1/16 in. | 3/32 in. (2.38 mm) |
| | 30% Lutrol ® F68 | (1.59 mm) | |

### EXAMPLE 4

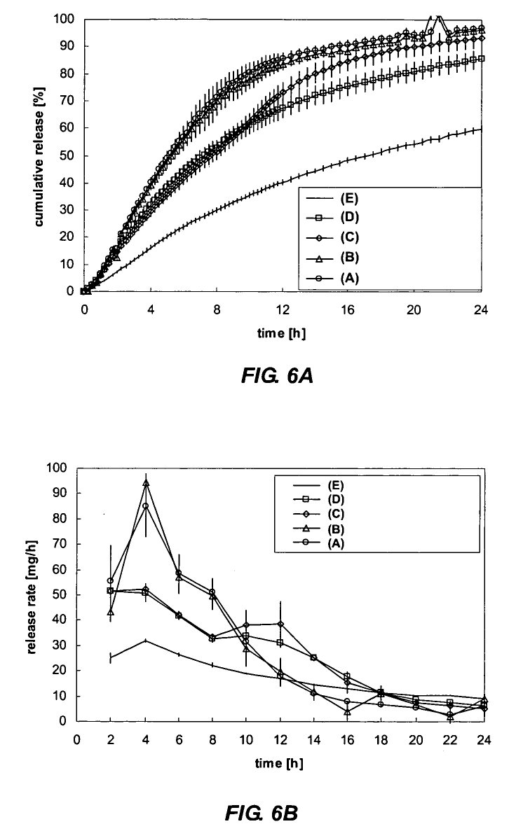

Five dosage forms, each having a structure similar to the one shown in FIGS. 2C and 2D, were prepared. Each dosage form had the formulation shown in Table 2 as the active agent composition. Each dosage form had a semipermeable membrane enclosing the active agent composition. The semipermeable membrane in each dosage form was composed of 70% by weight Eastman cellulose acetate (CA 398-10) and 30% by weight Lutrol® F68 and had a thickness of 2.5 mm. Each dosage form had eight squeeze bands circumscribing portions of the semipermeable membrane, and the squeeze bands were made of silicone elastomer. The study evaluated the effect of number and size of delivery orifice in the semipermeable membrane on release rate. Additional properties of the squeeze bands and delivery orifice(s) in each dosage form are shown in Table 5. FIGS. 6A and 6B show cumulative release and release rate, respectively, for the dosage forms. The results show that the delivery orifice size and number do not have a significant effect on the release rate.

TABLE 5

| | | | No. of | Size of |
|---|---|---|---|---|
| Dosage | Squeeze Band | Squeeze Band | Delivery | Delivery |
| Form | (Inner Diameter) | (Outer Diameter) | Orifices | Orifices |
| A | 1/16 in. | 1/8 in. (3.18 mm) | 2 | 30 mm |
| | (1.59 mm) | | | |
| B | 1/16 in. | 1/8 in. (3.18 mm) | 2 | 40 mm |
| | (1.59 mm) | | | |
| C | 1/16 in. | 3/32 in. (2.38 mm) | 1 | 40 mm |
| | (1.59 mm) | | | |
| D | 1/16 in. | 3/32 in. (2.38 mm) | 2 | 40 mm |
| | (1.59 mm) | | | |
| E | 1/4 in. | 5/16 in. (7.94 mm) | 2 | 40 mm |
| | (6.35 mm) | | | |

### EXAMPLE 5

Two dosage forms, each having a structure similar to the one shown in FIGS. 2C and 2D, were prepared. Each dosage form had the formulation shown in Table 6 as the active agent composition.

TABLE 6

| | Substance | Wt % |
|---|---|---|
| Active Agent | Acetaminophen (solubility = 20 mg/ml) | 90 |
| Hydrophilic | Polyox N750 | 7 |
| Polymer | | |
| Binder | PVP XL | 2 |
| Lubricant | Magnesium Stearate | 1 |

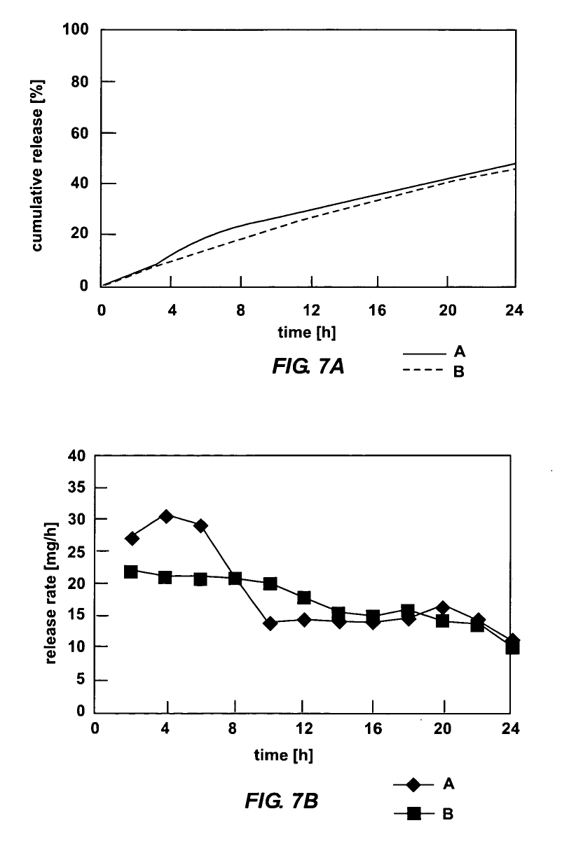

In Example 5, the semipermeable membrane in each dosage form had one 156-mm delivery orifice. The semipermeable membrane in each dosage form was composed of 70% by weight Eastman cellulose acetate (CA 398-10) and 30% by weight Lutrol® F68 and had a thickness of 2.5 mm. Each dosage form had eight squeeze bands circumscribing portions of the semipermeable membrane, and the squeeze bands were made of silicone elastomer. Additional properties of the squeeze bands used in the study are shown in Table 7. FIGS. 7A and 7B show cumulative release and release rate, respectively, of the dosage form as a function of band size. The dosage form showed no enhanced release due to squeeze effect because the active agent composition was difficult to hydrate. This formulation that was difficult to hydrate contained 90% by weight of acetaminophen (solubility=20 mg/ml). The formulations used in previous examples were not difficult to hydrate and contained 70% or 80% by weight acetaminophen.

TABLE 7

| Dosage | Squeeze Band | Squeeze Band | Wall |
|---|---|---|---|
| Form | (Inner Diameter) | (Outer Diameter) | Thickness |
| A | 1/16 in. (1.59 mm) | 1/8 in. (3.18 mm) | 1.59 mm |
| B | 1/4 in. (6.35 mm) | 5/16 in. (7.94 mm) | 1.59 mm |

### EXAMPLE 6

Four dosage forms, each having a structure similar to the one shown in FIGS. 2C and 2D, were prepared. Each dosage form had the formulation shown in Table 8 as the active agent composition.

TABLE 8

| | Substance | Wt % |
|---|---|---|
| Active Agent | Ranitidine (solubility = 880 mg/ml) | 89 |
| Binder | PVP | 7 |
| Lubricant | Magnesium Stearate | 4 |

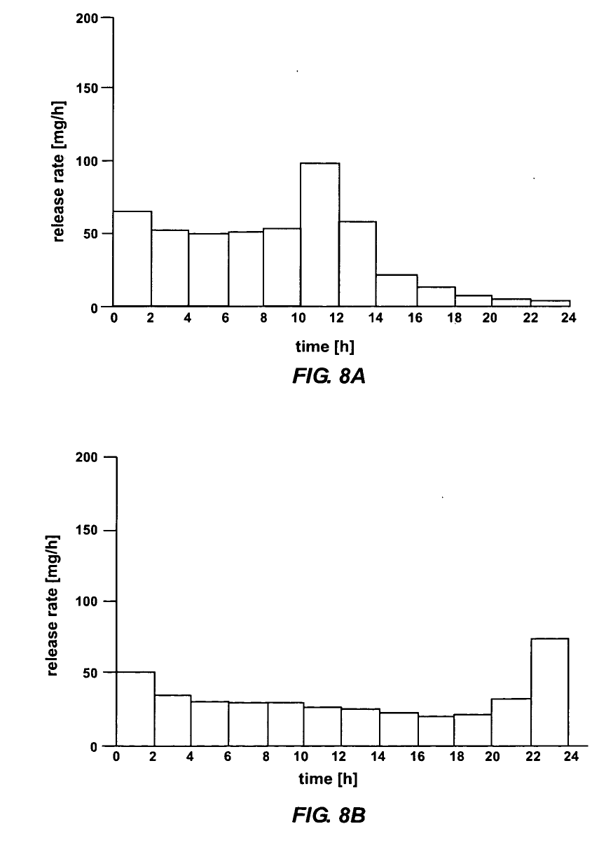

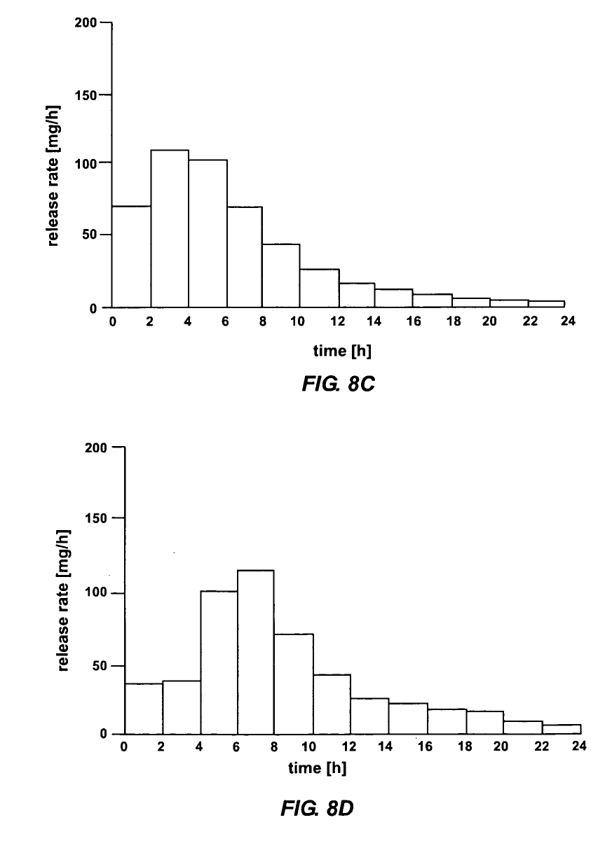

In Example 6, each dosage form had a semipermeable membrane enclosing the active agent composition. The semipermeable membrane in each dosage form had two 15-mm delivery orifices. The semipermeable membrane in each dosage form was composed of 95% by weight Eastman cellulose acetate (CA 398-10) and 5% by weight Lutrol® F68 and had a thickness of 2.5 mm. Three of the dosage forms had eight squeeze bands circumscribing portions of the semipermeable membrane, and the squeeze bands were made of silicone elastomer. Physical dimensions of the squeeze bands used in the dosage forms are summarized in Table 9 below. FIGS. 8A-8D show the release rate of the dosage forms. The results show that application of squeeze deviates from zero-order release.

TABLE 9

| Dosage | Squeeze Band | Squeeze Band | Wall |
|---|---|---|---|
| Form | (Inner Diameter) | (Outer Diameter) | Thickness |
| A | None | None | None |
| B | 1/4 (6.35 mm) | 5/16 (7.94 mm) | 1.59 mm |
| C | 1/8 (3.18 mm) | 1/4 (6.35 mm) | 3.17 mm |
| D | 1/16 (1.59 mm) | 1/8 (3.18 mm) | 1.59 mm |

The invention may provide one or more advantages. The dosage form of the present invention uses a squeeze layer made of a material that changes shape at physiological temperature to drive delivery of an active agent. The squeeze layer eliminates the need for an expandable composition inside the dosage form to push the active agent out of the dosage form. This makes the entire internal volume of the dosage form available for loading of an active agent, yielding a dosage form with a high active agent loading efficiency.

While the invention has been described with respect to a limited number of embodiments, those skilled in the art, having benefit of this disclosure, will appreciate that other embodiments can be devised which do not depart from the scope of the invention as disclosed herein. The following claims summarizes exemplary embodiments of the invention.

1. A controlled release dosage form, comprising:
an active agent composition; and
a squeeze layer circumscribing at least a portion of the active agent composition, the squeeze layer comprising a material that changes shape in a selected fluid environment of use, wherein the material when it changes shape applies a squeeze force to the active agent composition.

2. The controlled release dosage form of claim 1, wherein the material is sensitive to a temperature or pH of the selected fluid environment of use.

3. The controlled release dosage form of claim 1, wherein the squeeze layer forms a compartment that is occupied by the active agent composition.

4. The controlled release dosage form of claim 3, wherein the squeeze layer comprises one or more openings communicating the compartment to an exterior of the dosage form.

5. The controlled release dosage form of claim 1, further comprising a semipermeable membrane interposed between the active agent composition and the squeeze layer.

6. The controlled release dosage form of claim 5, wherein the semipermeable membrane forms a compartment that is occupied by the active agent composition and the squeeze layer applies the squeeze force to both the semipermeable membrane and the active agent composition.

7. The controlled release dosage form of claim 6, wherein the semipermeable membrane comprises one or more delivery orifices communicating the compartment to an exterior of the dosage form.

8. The controlled release dosage form of claim 6, wherein the squeeze layer comprises one or more bands, each band circumscribing a portion of the semipermeable membrane and applying a squeeze force to the semipermeable membrane which is transferred to the active agent composition.

9. The controlled release dosage form of claim 1, wherein the squeeze layer comprises one or more bands, each band applying a squeeze force to the active agent composition.

10. The controlled release dosage form of claim 1, wherein the material that changes shape is selected from the group consisting of thermoplastic elastomers, shape memory polymers, and their copolymers.

11. The controlled release dosage form of claim 1, wherein the active agent composition comprises an active agent that provides a pharmacologic effect.

12. The controlled release dosage form of claim 11, wherein the active agent composition is formulated such that when it is hydrated it forms a wet mass in which the active agent dissolves or suspends.

13. The controlled release dosage form of claim 12, wherein the active agent further comprises a hydrophilic polymer.

14. A method of delivering an active agent to a fluid environment of use, comprising:
placing a controlled release dosage form in the fluid environment of use, the controlled release dosage form comprising an active agent composition including the active agent and a squeeze layer circumscribing at least a portion of the active agent composition, the squeeze layer comprising a material that changes shape in the fluid environment of use, wherein the material when it changes shape generates a squeeze force;
hydrating the active agent composition such that a wet mass is formed in which the active agent either dissolves or suspends; and
applying the squeeze force to the active agent composition to deliver the active agent.
